(19) United States
(12) Patent Application Publication
(10) Pub. No.: US 2026/0342754 A1
(43) Pub. Date: Oct. 29, 2026

(54) METHODS AND SYSTEMS FOR PRIORITIZING COMMUNICATIONS BASED ON COMMUNICATION ATTRIBUTES

(71) Applicant: Sequelae, Inc., Minneapolis, MN (US)

(72) Inventors: Alex Ruch, Prosper, TX (US); Natalie Sheils, St. Paul, MN (US); Sara Taylor, Edina, MN (US); Salomea Anna Jankovic, Minneapolis, MN (US); Duncan Hieu Minh Dam, Palm Springs, CA (US)

(73) Assignee: Sequelae, Inc., Minneapolis, MN (US)

(21) Appl. No.: 19/661,986

(22) Filed: Apr. 29, 2026

Related U.S. Application Data
(60) Provisional application No. 63/796,706, filed on Apr. 29, 2025.

(51) Int. Cl. H04L 51/02; G06F 40/20; G16H 50/80

(52) U.S. Cl. CPC H04L 51/02; G06F 40/20; G16H 50/80

(57) ABSTRACT

Methods and systems for prioritizing communications based on communication attributes are provided. A digital communication network (DCN) may be provided for a plurality of members of a population to interact with each other and/or the DCN. A plurality of communications from the DCN are analyzed and one or more attributes are determined for each communication. At least one communication is prioritized for displaying to an individual member based on the one or more attributes.

### CROSS REFERENCE TO RELATED APPLICATIONS

This application claims the benefit of U.S. Provisional Application No. 63/796,706, filed on April 29, 2025, which application is incorporated herein by reference in its entirety.

### BACKGROUND

Various embodiments relate generally to prioritizing communications in a digital communication network (DCN) and more particularly to prioritizing communications based on one or more attributes of the communications.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Digital networks, which may include the use of social media, allow individuals to interact with others online and connect many people with a community. The online community can be utilized to help build positive behaviors and encourage people to make improvements in their lives.

What is needed is a way to prioritize communications to a member of the population or the population as a whole, based on one or more interdependent attributes of the communications, with the aid of computers and online communities.

### SUMMARY

Example aspects of the present disclosure include:

In at least one embodiment of the present disclosure, a method to slow a progression of an adverse condition in members of a population comprises providing a digital communication network (DCN) for a plurality of members of a population to interact with each other and the DCN; analyzing a plurality of communications in the DCN; determining one or more attributes of each communication of the plurality of communications, the one or more attributes comprising a degree of usefulness and a degree of spread for the plurality of communications, wherein the degree of usefulness correlates to the degree to which a communication of the plurality of communications contains an idea that is useful in slowing the progression of the adverse condition and the degree of spread correlates to the degree to which the idea will spread in the digital communication network; and prioritizing displaying at least one communication of the plurality of communications to the individual member based on the one or more attributes.

Any of the aspects herein, wherein analyzing the plurality of communications includes automatically determining the idea.

Any of the aspects herein, wherein the idea comprises at least one of an action or an experience undertaken and the motivation behind undertaking the action.

Any of the aspects herein, wherein analyzing the plurality of communications includes automatically creating an ontology and associating at least one of the idea or the motivation with the ontology.

Any of the aspects herein, wherein a language model executes the analyzing the plurality of communications.

Any of the aspects herein, wherein the language model is a large language model.

Any of the aspects herein, wherein analyzing the plurality of communications includes determining the adverse condition associated with an individual who created the communication.

Any of the aspects herein, wherein analyzing the plurality of communications includes automatically creating an ontology and associating at adverse condition with the ontology.

Any of the aspects herein, wherein the ontology comprises a sematic graph.

Any of the aspects herein, wherein the condition is at least one of chronic systemic inflammation, malaise, social dysfunction, or low energy.

Any of the aspects herein, wherein the condition is a disease.

Any of the aspects herein, wherein the disease is at least one of: a metabolic disease, such as obesity, diabetes, cardiovascular disease, complement factor H, chronic kidney disease, and nonalcoholic steatohepatitis; an autoimmune disease, such as, rheumatoid arthritis, Crohn’s disease, eczema, psoriasis, psoriatic arthritis, and type 1 diabetes; an inflammation moderated lung disease, such as, chronic obstructive pulmonary disease and asthma, and an oncological disease.

Any of the aspects herein, wherein the condition is a prodromal version of the disease.

Any of the aspects herein, wherein the condition is a health risk.

Any of the aspects herein, wherein the health risk comprises at least one of falling or becoming infected.

Any of the aspects herein, wherein a conversation bot paraphrases the prioritizes communication delivers the communication to the individual member in a paraphrased form.

Any of the aspects herein, wherein the communication in the DCN between members of the population is at least one of one to one, one to many, one to system, system to one, or a system to many.

Any of the aspects herein, wherein the system comprises a conversation bot that derives its communications from at least one of the analysis of communication in the digital communication network or biometrics provided to the DCN.

Any of the aspects herein, wherein the method is used to at least one of manage a disease, lower health risk in a population, and manage health care costs.

A method to slow a progression of an adverse condition in members of a population according to at least one embodiment of the present disclosure comprises providing a digital communication network (DCN) for a plurality of members of a population to interact with each other and the DCN; analyzing a plurality of communications in the DCN; determining one or more attributes of each communication of the plurality of communications, the one or more attributes comprising a degree of usefulness and a degree of spread for the plurality of communications, wherein the degree of usefulness correlates to the degree to which a communication of the plurality of communications contains an idea that is useful in slowing the progression of the adverse condition and the degree of spread correlates to the degree to which the idea will spread in the digital communication network; generating an ontology comprising a semantic graph with a plurality of edges connecting one or more nodes, wherein each node is assigned at least one of an idea embedding correlating to the idea, an adverse condition embedding correlating to the adverse condition, or an attribute embedding correlating to the attribute, and wherein each edge defines a relationship between connected nodes; and prioritizing displaying at least one communication of the plurality of communications to the individual member based on ontology.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3A shows an example of a computing system, in accordance with one or more embodiments.

FIG. 3B shows an example of a network, in accordance with one or more embodiments.

### DETAILED DESCRIPTION

Understanding a contagion of ideas in a community or population requires knowledge about the attributes of the ideas and the structure of the community. In at least one embodiment of the present disclosure, the attributes of an idea or a member or the population are not treated as fixed quantities but rather are treated as interdependent attributes. In at least one aspect, the interdependency is mediated by the member’s motivation for interacting with the idea. The motivation is also important in understanding contagion as the attributes of the idea itself.

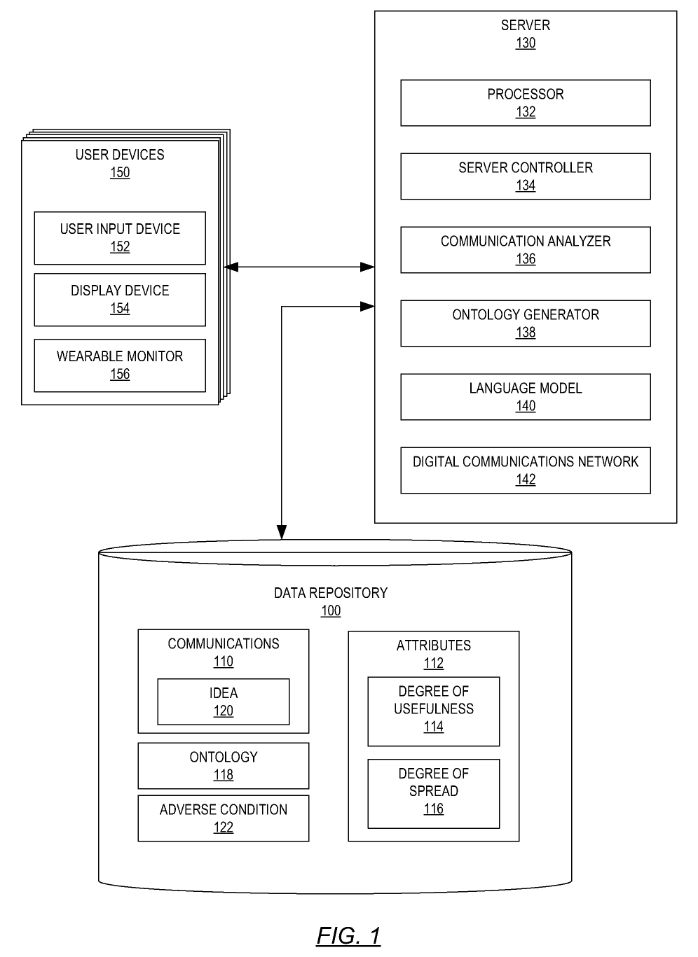

Turning to the Figures, the system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communications (110). The communications (110) are communications from members in a digital communication network (DCN) (142) (described in more detail below). The communications (110) may be, for example, text-based, image-based, multimedia communications, audio communications, or any combinations thereof. The communications may be between members, communications in a forum, communications with the DCN (142), or communications generated by the DCN (142) (e.g., communications from a chatbot).

The communications (110) include an idea (120) that is useful in slowing the progression of the adverse condition (122). The idea (120) includes an action, or an experience undertaken by the individual member who created the communication, and the motivation behind undertaking the action. For example, the adverse condition (122) may be, for example, low energy, the idea (120) may be to walk a predetermined amount of time each day, and the motivation may be to increase the individual member’s low energy by walking.

In some embodiments, the adverse condition (122) is chronic systemic inflammation, malaise, social dysfunction, low energy, a disease, and/or a health risk. In embodiments where the adverse condition is a disease, the disease may be a metabolic disease, such as obesity, diabetes, cardiovascular disease, chronic kidney disease, and nonalcoholic steatohepatitis; an autoimmune disease, such as, rheumatoid arthritis, Crohn’s disease, eczema, psoriasis, psoriatic arthritis, and type 1 diabetes; an inflammation moderated lung disease, such as, chronic obstructive pulmonary disease and asthma, or an oncological disease. The adverse condition may be a prodromal version of the disease. In embodiments where the adverse condition is a health risk, the health risk may be, for example, falling or becoming infected.

The data repository (100) also stores attributes (112). The attributes (112) are attributes (112) of the communications (110) that can be used to understand the contagion of ideas (120) in a community. The attributes (112) may be, for example, interdependent on each other. The attributes (112) may include, for example, a degree of usefulness (114) and a degree of spread (116). More specifically, the degree of usefulness correlates to the degree to which a communication (110) (of a plurality of communications) contains an idea (120) that is useful in slowing the progression of the adverse condition (122). The degree of spread (116) correlates to the degree to which the idea will spread in the DCN (142).

The data repository (100) also stores an ontology (118). The ontology (118) is a defined set of relationships between the adverse condition (122), ideas (120), motivations, and/or attributes (112) and how the adverse condition (122), ideas (120), motivations, and/or attributes (112) relate to each other. The ontology (118) is used to define and show the interdependency of the attributes (118) and/or ideas (120).

The ontology (118) may be, for example, a semantic graph with nodes and edges. In such example, the attributes (112) may be encoded into attribute embeddings and stored in the nodes. Additionally, the ideas (120) may be encoded into idea embeddings, and the adverse conditions (122) may be encoded into adverse condition embeddings and stored in the nodes. The nodes may be linked by the edges, which represent relationships between the nodes. For example, nodes that are near each other in space and connected by an edge have some relation to each other.

Encoding the attribute embeddings, adverse condition embeddings, and/or idea embeddings may include converting the attributes (112), the adverse conditions (122), and/or ideas (120) into vectors that capture their semantic meaning. Such conversion may be performed by, for example, an ontology generator (138), described in more detail below. Attributes (112), adverse conditions (122), and/or ideas (120) that are semantically similar may have similar vectors. For example, a first idea may be jogging and a second idea may be walking. The first idea and the second idea may have similar vectors reflecting that they are both related to aerobic activities that involve moving one’s feet.

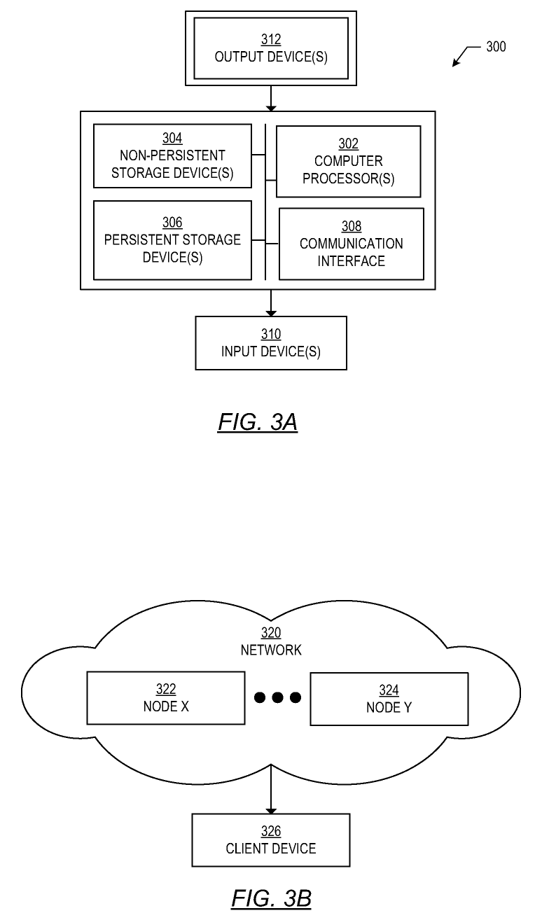

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136) and an ontology generator (138). An example of a computer system and network that may form the server (130) is described with respect to FIG. 3A and FIG. 3B.

The server (130) also includes a computer processor (132). The computer processor (132) is one or more hardware or virtual processors which may execute computer readable program code that defines one or more applications, such as the communication analyzer (136) and the ontology generator (138). An example of the computer processor (132) is described with respect to the computer processor(s) (302) of FIG. 3A.

The server (130) also may include a server controller (134). The server controller (134) is software or application specific hardware which, when executed by the computer processor (302), controls and coordinates operation of the software or application specific hardware described herein. Thus, the server controller (134) may control and coordinate execution of the communication analyzer (136) and the ontology generator (138).

The server (130) also includes the communication analyzer (136). The communication analyzer (136) is software or application specific hardware which, when executed by the computer processor (132), determines and/or prioritizes communications (110) in the DCN (142) to present an individual member based on the attributes (112) of the communications (110). For example, the communication analyzer (136) may receive the attributes (112), the communications (110), and information about the individual member and may output the communications (110) in a prioritized order. For example, the communications (110) may be prioritized based on the attributes (112) that correlate to the individual member’s adverse condition.

The server (130) also includes the ontology generator (138). The ontology generator (138) is software or application specific hardware which, when executed by the computer processor (132), generates a definition or defines a relationship to associate adverse condition, ideas, motivations, and/or attributes (112) to the ontology (118).

In some embodiments, the ontology generator (138) is configured to convert ideas (120), adverse conditions (122), and/or attributes (112) into idea embeddings, adverse condition embeddings, and/or attribute embeddings, respectively, and to generate a semantic graph. As previously described, the idea embeddings, adverse condition embeddings, and/or attribute embeddings are assigned to nodes and the ontology generator (138) may further connect nodes by edges that define relationships between connected nodes.

The server (130) also includes a language model (140). The language model (140) is a natural language processing machine learning model. An example of the language model (140) may be a large language model (LLM), such as a CHATGPT^{®} LLM or LLAMA^{®} LLM. However, many different language models may be used. In some embodiments, the language model (140) can be used to analyze the communications (110), generate the ontology (118), and/or associate the attributes (112), ideas, and/or motivations with the ontology.

In some embodiments, the communication analyzer (136) and/or the ontology generator (138) may be a language model such as the language model (140). In still other embodiments, the communication analyzer (136) and/or the ontology generator (138) may be agents. Agents are a computer implemented software component that can interface and/or include a language model such as the language model (140) and autonomously generates, selects, and executes one or more actions based on language model outputs, system state, and objective criteria, without requiring continuous human control. The agent may be configured to ingest inputs, consult with the language model, select and execute actions, and maintain state information. The agent may further be configured to evaluate outcomes and update itself in a feedback mechanism.

The server (130) also includes the digital communications network (DCN) (142). The DCN (142) is a network through which members of a population can interact with each other, or with a system supported by the DCN (142). The DCN (142) can also communicate or otherwise deliver the communications (110) in the prioritized order to individual members of the population. The communication (110) in the DCN (142) between members of the population can be one to one, one to many, one to system, system to one, or a system to many. The DCN may also be, for example, a conversation bot.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156) and be configured to send, for example, biometrics, the communications (110), or any other input to the server (130). In an alternative embodiment, a separate wearable device may be in communication with the user device (150), such as a smart watch, or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

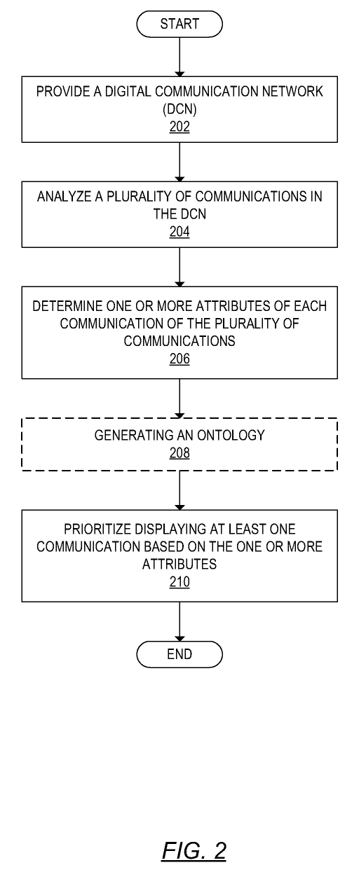

FIG. 2 is a logic flow diagram that illustrates a method, and a result of execution of computer program instructions, in accordance with various embodiments. The method can be used to prioritize communication(s), such as the communications (110) based on attribute(s), such as the attribute(s) (112) of the communications.

At Block 202, a step of providing a digital communication network (DCN) is provided. The DCN may be the same as or similar to the DCN (142) and provides a network for members of a population to interact with each other or with the DCN (142).

At Block 204, a step of analyzing a plurality of communications in the DCN is provided. The communications may be the same as or similar to the communications (110) and can be analyzed by, for example, a communication analyzer, such as the communication analyzer (136). The communication analyzer (136) receives at least the communications and may receive information about the member who created the communication and outputs one or more attributes, such as the attributes (112), as described below. In other embodiments, the plurality of communications may be analyzed by a language model, such as the language model (140) which may be, for example, a large language model.

At Block 206, a step of determining one or more attributes of the plurality of communications is provided. The one or more attributes may be determined by, for example, the communication analyzer. As previously described, the one or more attributes includes at least a degree of usefulness and a degree of spread for the plurality of communications. The degree of usefulness correlates to the degree to which a communication of the plurality of communications contains an idea that is useful in slowing the progression of the adverse condition and the degree of spread correlates to the degree to which the idea will spread in the digital communication network. The idea and/or the adverse condition may be determined automatically by, for example, the communication analyzer. The idea may include at least one of an action or an experience undertaken and the motivation behind undertaking the action.

The attributes described herein are not fixed quantities but are interdependent. In at least one aspect, the interdependency is mediated by the member’s motivation for interacting with the idea. For example, the spread of the idea that continuous glucose monitoring (CGM) is an easy way to understand one’s lifestyle is mediated by whether the individual member seeing the idea is motivated by disease management, weight loss, social position in the network. Thus, motivation is also important in understanding contagion as the attributes of the idea itself.

At Block 206, a step of generating an ontology is provided. The Block 206 may be optional in some embodiments. An ontology generator such as the ontology generator (138) may generate an ontology, such as the ontology (118) and associate the idea, motivation, and/or adverse condition with the ontology. In other embodiments, the ontology may be generated by the language model which may be, for example, the large language model. The ontology can be developed to beneficially describe health related actions and experiences and the motivations to undertake them and other related aspects required to optimize the spread of useful ideas.

In some embodiments, the ontology is a semantic graph with nodes and edges. More specifically, the ontology generator may convert or encode the ideas, the adverse conditions, and/or the attributes into idea embeddings, adverse condition embeddings, and/or attribute embeddings. The ideas (120) idea embeddings, adverse condition embeddings, and/or attribute embeddings are assigned to nodes and connected via edges, which define relationships between connected nodes.

At Block 208, a step of prioritizing the communications and displaying at least one prioritized communication based on the one or more attributes is provided. More specifically, the at least one communication may be prioritized based on the level of degrees of usefulness and spread. For example, communications with higher degrees of usefulness to the individual may be prioritized over communications with lower degrees of usefulness. In some embodiments, a conversation bot supported by the DCN paraphrases the prioritizes communication and delivers the communication to the individual member in a paraphrased form.

In some embodiments, prioritizing the communications includes using the ontology graph to determine which communications to prioritize. For example, ideas, attributes, and/or an adverse condition extracted from the communications can be used to search the ontology for relationships between the ideas, attributes, and/or adverse condition that are graphically near each other or connected by edges. Such information may correlate to related communications that can be grouped and/or prioritized together.

The method described above in FIG. 2 can include more or less steps. Further, the steps can be in any order and any step or combination of steps can be repeated.

The method and systems described herein can be beneficially used to slow a progression of an adverse condition in a member of the population by prioritizing communications to the member based on interdependent attributes of the communications. The methods and systems can also beneficially be used to manage a disease, lower health risk in a population, and manage health care costs. The methods and systems further improve the effectiveness of communications in a DCN for, for example, improving health and wellbeing in an individual member and the population.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 3A, the computing system (300) may include one or more computer processor(s) (302), non-persistent storage device(s) (304), persistent storage device(s) (306), a communication interface (308) (e.g., Bluetooth interface, infrared interface, network interface, optical interface, etc.), and numerous other elements and functionalities that implement the features and elements of the disclosure. The computer processor(s) (302) may be an integrated circuit for processing instructions. The computer processor(s) (302) may be one or more cores, or micro-cores, of a processor. The computer processor(s) (302) includes one or more processors. The computer processor(s) (302) may include a central processing unit (CPU), a graphics processing unit (GPU), a tensor processing unit (TPU), combinations thereof, etc.

The input device(s) (310) may include a touchscreen, keyboard, mouse, microphone, touchpad, electronic pen, or any other type of input device. The input device(s) (310) may receive inputs from a user that are responsive to data and messages presented by the output device(s) (312). The inputs may include text input, audio input, video input, etc., which may be processed and transmitted by the computing system (300) in accordance with one or more embodiments. The communication interface (308) may include an integrated circuit for connecting the computing system (300) to a network (not shown) (e.g., a local area network (LAN), a wide area network (WAN), such as the Internet, mobile network, or any other type of network) or to another device, such as another computing device, and combinations thereof.

Further, the output device(s) (312) may include a display device, a printer, external storage, or any other output device. One or more of the output device(s) (312) may be the same or different from the input device(s) (310). The input device(s) (310) and output device(s) (312) may be locally or remotely connected to the computer processor(s) (302). Many different types of computing systems exist, and the aforementioned input device(s) (310) and output device(s) (312) may take other forms. The output device(s) (312) may display data and messages that are transmitted and received by the computing system (300). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium, such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (302), is configured to perform one or more embodiments, which may include transmitting, receiving, presenting, and displaying data and messages described in the other figures of the disclosure.

The computing system (300) in FIG. 3A may be connected to, or be a part of, a network. For example, as shown in FIG. 3B, the network (320) may include multiple nodes (e.g., node X (322) and node Y (324), as well as extant intervening nodes between node X (322) and node Y (324)). Each node may correspond to a computing system, such as the computing system shown in FIG. 3A, or a group of nodes combined may correspond to the computing system shown in FIG. 3A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (300) may be located at a remote location and connected to the other elements over a network.

The nodes (e.g., node X (322) and node Y (324)) in the network (320) may be configured to provide services for a client device (326). The services may include receiving requests and transmitting responses to the client device (326). For example, the nodes may be part of a cloud computing system. The client device (326) may be a computing system, such as the computing system shown in FIG. 3A. Further, the client device (326) may include or perform all or a portion of one or more embodiments.

The computing system of FIG. 3A may include functionality to present data (including raw data, processed data, and combinations thereof), such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method to slow a progression of an adverse condition in members of a population, the method comprising:
providing a digital communication network (DCN) for a plurality of members of a population to interact with each other and the DCN;
analyzing a plurality of communications in the DCN;
determining one or more attributes of each communication of the plurality of communications, the one or more attributes comprising a degree of usefulness and a degree of spread for the plurality of communications, wherein the degree of usefulness correlates to the degree to which a communication of the plurality of communications contains an idea that is useful in slowing the progression of the adverse condition and the degree of spread correlates to the degree to which the idea will spread in the digital communication network; and
prioritizing displaying at least one communication of the plurality of communications to the individual member based on the one or more attributes.

2. The method of claim 1, wherein analyzing the plurality of communications includes automatically determining the idea.

3. The method of claim 1, wherein the idea comprises at least one of an action or an experience undertaken and the motivation behind undertaking the action.

4. The method of claim 3, wherein analyzing the plurality of communications includes automatically creating an ontology and associating at least one of the idea or the motivation with the ontology.

5. The method of claim 1, wherein a language model executes the analyzing the plurality of communications.

6. The method of claim 5, wherein the language model is a large language model.

7. The method of claim 1, wherein analyzing the plurality of communications includes determining the adverse condition associated with an individual who created the communication.

8. The method of claim 1, wherein analyzing the plurality of communications includes automatically creating an ontology and associating at adverse condition with the ontology.

9. The method of claim 8, wherein the ontology comprises a sematic graph.

10. The method of claim 1, wherein the condition is at least one of chronic systemic inflammation, malaise, social dysfunction, or low energy.

11. The method of claim 1, wherein the condition is a disease.

12. The method of claim 11, wherein the disease is at least one of:
a metabolic disease, such as obesity, diabetes, cardiovascular disease, complement factor H, chronic kidney disease, and nonalcoholic steatohepatitis;
an autoimmune disease, such as, rheumatoid arthritis, Crohn’s disease, eczema, psoriasis, psoriatic arthritis, and type 1 diabetes;
an inflammation moderated lung disease, such as, chronic obstructive pulmonary disease and asthma, and
an oncological disease.

13. The condition of claim 11, wherein the condition is a prodromal version of the disease.

14. The method of claim 1, wherein the condition is a health risk.

15. The method of claim 14, wherein the health risk comprises at least one of falling or becoming infected.

16. The method of claim 1, wherein a conversation bot paraphrases the prioritizes communication delivers the communication to the individual member in a paraphrased form.

17. The method of claim 1, wherein the communication in the DCN between members of the population is at least one of one to one, one to many, one to system, system to one, or a system to many.

18. The method of claim 17, wherein the system comprises a conversation bot that derives its communications from at least one of the analysis of communication in the digital communication network or biometrics provided to the DCN.

19. The method of claim 1, wherein the method is used to at least one of manage a disease, lower health risk in a population, and manage health care costs.

20. A method to slow a progression of an adverse condition in members of a population, the method comprising:
providing a digital communication network (DCN) for a plurality of members of a population to interact with each other and the DCN;
analyzing a plurality of communications in the DCN;
determining one or more attributes of each communication of the plurality of communications, the one or more attributes comprising a degree of usefulness and a degree of spread for the plurality of communications, wherein the degree of usefulness correlates to the degree to which a communication of the plurality of communications contains an idea that is useful in slowing the progression of the adverse condition and the degree of spread correlates to the degree to which the idea will spread in the digital communication network;
generating an ontology comprising a semantic graph with a plurality of edges connecting one or more nodes, wherein each node is assigned at least one of an idea embedding correlating to the idea, an adverse condition embedding correlating to the adverse condition, or an attribute embedding correlating to the attribute, and wherein each edge defines a relationship between connected nodes; and
prioritizing displaying at least one communication of the plurality of communications to the individual member based on ontology.
